(19) United States
(12) Patent Application Publication
(10) Pub. No.: US 2004/0015071 A1
(43) Pub. Date: Jan. 22, 2004

(54) Magnetic resonance imaging apparatus

(76) Inventors: Kazumi Komura, Matsudo-shi, Chiba (JP); Tetsuhiko Takahashi, Souka-shi, Saitama (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/344,372

(22) PCT Filed: Aug. 10, 2001
(86) PCT No.: PCT/JP01/06910

(30) Foreign Application Priority Data
Aug. 11, 2000 (JP) 2000-244219

(51) Int. Cl. A61B 5/05

(52) U.S. Cl. 600/410

(57) ABSTRACT

To efficiently generate an accurate morphological image and the temperature change distribution image, a pulse sequence for acquiring a plurality of echo signals having different echo times is executed, while excited spins are encoded with the same phase. Among thus obtained plural echo signals, the echo signal 405 acquired in the echo time TE1 suitable for obtaining morphological information (anatomic information) is used to reconstruct a morphological image. Further, the PPS method is applied to the echo signal 406 acquired in the echo time TE2 suitable for thermometry so as to generate the temperature change distribution image. The echo signal used for generating the morphological image may be a spin echo signal or a gradient echo signal.

### FIELD OF THE INVENTION

The present invention relates to a technique for obtaining morphological information (anatomic information) of an object to be examined and the temperature distribution within the object by using a magnetic resonance imaging apparatus.

### BACKGROUND OF THE INVENTION

A magnetic resonance imaging (hereinafter referred to as MRI) apparatus measures density distribution, relaxation time distribution and the like of nuclear spins in a desired diagnostic region in the object to be examined by utilizing magnetic resonance phenomenon, and then displays a cross-sectional image of the object using thus measured data.

In recent years, the interventional MRI referred to as IV-MRI, in which an MRI apparatus is used as a monitor while conducting treatment, has been attracting attention. Known methods of treatment using the IV-MRI include laser treatment, treatment by drug injection using drugs such as ethanol, excision with RF radiation, and low-temperature treatment. In those treatment methods, the MRI apparatus is used for guiding a needle or tubule to a lesion by performing real-time imaging, for visualizing the physiological changes during treatment, for monitoring temperature in the examined region during heating or cooling treatment, and for imaging the temperature distribution of a body in laser treatment.

On the other hand, as methods of measuring the temperature distribution in an object utilizing an MRI apparatus, there are known a signal intensity method in which the temperature distribution is calculated from nuclear magnetic resonance (NMR) signal intensity, a proton phase shift (PPS) method in which the temperature distribution is calculated from the phase shift of NMR signals, and a method utilizing the diffusion coefficient of NMR signals, a coefficient that depends on the temperature.

Hereinafter, calculation of the temperature distribution utilizing the PPS method will be described in detail, with reference to the calculation with phase information of gradient echo signals.

As shown in FIG. 7, in a gradient echo pulse sequence, a slice-select gradient magnetic field Gs102 and 90° radio frequency (RF) pulse RF101 are applied to the object in accordance with the slicing position, thus exiting the nuclear spins of the slice. Then, a phase encoding gradient magnetic field Gp103 and a frequency-encoding/readout gradient magnetic field Gr104 are applied so as to generate and detect encoded gradient echo signals 105 which provide positional information within the slice. This pulse sequence is repeated, while the phase encoding gradient magnetic field Gp103 is gradually changed.

Then, from the a real part Sr(x,y) and an imaginary part Si(x,y) of a complex image that are calculated by performing two-dimensional Fourier transformation on the detected gradient echo signals, the phase distribution φ(x,y) is calculated in accordance with, for example, formula (1):

$$φ ( x , y ) = {tan}^{- 1} ( \frac{Si ( x , y )}{Sr ( x , y )} ) \qquad ( 1 )$$

And, the temperature distribution T(x,y) is calculated from the above-calculated spatial phase distribution, the time interval (the echo time) TE (ms) between the time point when a 90° RF pulse RF101 is applied and the time point when the gradient echo signal reaches its maximum value, a resonance frequency f (Hz), and the temperature coefficient of water −0.01 (ppm/° C.), in accordance with, for example, formula (2):

$$T = \frac{φ}{TE \cdot f \cdot - 0.01 \times {10}^{- 6} \cdot 360} \qquad ( 2 )$$

Next, the principle of measurement of the temperature distribution due to the signal intensity method will be described with reference to the calculation utilizing the phase information of the gradient echo signal.

The signal intensity S of the gradient echo signal acquired by repeating the gradient echo type pulse sequence described in FIG. 7 can be calculated by the formula (3), using the repetition time TR, the echo time TE, the vertical relaxation time T1, the transverse relaxation time T2, the flip angle α, and the magnetization intensity M:

$$S = M \frac{sin ( α ) \cdot ( 1 - exp ( - \frac{TR}{T1} ) )}{1 - cos ( α ) \cdot exp ( - \frac{TR}{T1} )} exp ( - \frac{TR}{{T2}^{*}} ) \qquad ( 3 )$$

Here, the vertical relaxation time T1 changes according to the temperature. For example, the change of T1 with temperature in liver tissue is 2.5 ms/° C. Therefore, the signal intensity due to the formula (3) depends on the temperature, and thus the brightness of a morphological image generated by an MRI apparatus also changes due to this signal intensity. That is, when the temperature rises in a region, the signal intensity of the gradient echo signal there becomes weak. Thus, in the morphological image displayed on the MRI apparatus in accordance with the gradient echo signal, the region in which the temperature rises is displayed darker than other region. Therefore, the temperature change in the object can be grasped to some extent by observing the morphological image obtained with the signal intensity method.

However, since the temperature dependency of T1 varies with tissues, it is hard to read the temperature distribution needed for treatment from such a morphological image.

On the other hand, the temperature distribution can be calculated more accurately by using the above-mentioned PPS method. However, since the echo time suitable for temperature measurement is determined by the thermal sensitivity of the tissue being examined or the range of measured temperature, said echo time Is not generally suitable for obtaining a morphological image. Concretely, in an MRI apparatus with 0.3T, when TE=30, 20, and 10 ms, the temperature change according to the phase change 1° are 0.71, 1.09, 2.17° C. respectively, and the range of measurable temperature is 130.2, 195.3, 390.6° C. respectively. Thus, the accuracy of the temperature measurement is improved as TE becomes longer.

However, for acquisition of a morphological image (anatomic information), shorter TE is preferable since S/N ratio thereby becomes high. That is, the desired condition for calculation of the temperature distribution is opposite to that for acquisition of a morphological image. Therefore, both the morphological image and the temperature distribution image can be preferably obtained by separately executing a pulse sequence for obtaining the morphological image and a pulse sequence for the temperature distribution with the echo times favorable for each of them. However, this method prolongs the operation time, and the lag behind real time is increased.

Due to the above-described reasons, it is difficult to perform measurement of the temperature distribution for said IV-MRI. Further, the efficiency is deteriorated because of the increase of processing load.

Therefore, the object of the present invention is to provide an MRI apparatus that can obtain both a morphological image and an image showing the temperature distribution or the temperature change distribution, accurately and efficiently.

### SUMMARY OF THE INVENTION

To achieve said object, an MRI apparatus of the present invention comprises:

static magnetic field generating means for generating a static magnetic field in a space in which an object to be examined is laid;

RF pulse generating means for applying an RF pulse to generate nuclear magnetic resonance in nuclear spins existing in a region of the object to be examined which has been laid in said static magnetic field;

gradient magnetic field generating means for applying to examined region a plurality of gradient magnetic fields including a phase encoding gradient magnetic field for phase-encoding an NMR signal generated in said examined region;

control means for controlling the application of said RF pulse and gradient magnetic fields to repeatedly execute the pulse sequence, in which a plurality of NMR signals having different echo times under the same phase encoding are generated after said nuclear spin is excited one time;

detecting means for detecting said NMR signals generated from the region with different respective echo times;

temperature distribution image generating means for generating the temperature distribution image of said region, using the NMR signals detected in a first echo time by said detecting means;

morphological image generating means for generating a morphological image of the examined region, using the NMR signals detected in a second echo time by said detecting means; and

image display means for displaying said temperature distribution image and said morphological image.

Further, in this MRI apparatus, said temperature distribution image generating means includes means for making an image of the temperature distribution in the examined region in accordance with a spatial phase distribution calculated with the NMR signals detected by said detecting means in said first echo time.

Further, in this MRI apparatus, said morphological image generating means comprises means for making a morphological image of the examined region using NMR signals detected in said first and the second echo time by said detecting means.

Further, in this MRI apparatus, said image display-means includes means for displaying both said temperature distribution image and said morphological image on one display. It is also possible to provide said image display means with means for inserting the temperature distribution of said region or inserting a temperature distribution image of the region where the temperature distribution is measured into said morphological image displayed on the full screen.

The pulse sequence executed in the present invention is a gradient echo type pulse sequence in which the RF pulse is applied one time, and then a plurality of readout gradient magnetic fields are applied with alternating polarity.

Further, the pulse sequence executed in the present invention may be the spin echo type pulse sequence in which a first RF pulse followed by a second RF pulse for inverting nuclear spins exited by the first RF pulse are applied, and then a plurality of readout gradient magnetic fields are applied with alternating polarity.

Further, to achieve said object, an MRI apparatus of the present invention comprises:

static magnetic field generating means for generating a static magnetic field in a space in which an object is laid;

RF pulse generating means for applying an RF pulse to generate nuclear magnetic resonance in the nuclear spins existing in an region to be examined of the object which has been laid in said static magnetic field;

gradient magnetic field generating means for applying to said examined region a plurality of gradient magnetic fields including a phase encoding gradient magnetic field for phase-encoding the NMR signals generated from said examined region;

control means for repeatedly operating the pulse sequence in which a plurality of NMR signals having different echo times generated under the same phase encoding by controlling the application of said RF pulse and gradient magnetic fields after exciting the nuclear spins one time, in order to time-sequentially perform imaging on said region of the object plural times;

detecting means for detecting the plurality of NMR signals having different echo times generated from said examined region in each imaging cycle;

temperature change distribution image generating means for generating the temperature change distribution image of said region by calculating the temperature distribution in said region using the NMR signals detected by said detecting means in a first echo time in each imaging cycle, and comparing one temperature distribution with others;

morphological image generating means for generating a morphological image of said examined region by using the NMR signals detected by said detecting means in a second echo time in one imaging; and

image display means for displaying said temperature change distribution image and morphological image.

In this MRI apparatus, said temperature change distribution image generating means includes means for making an image of the temperature change distribution in said region according to the spatial phase distribution, which is calculated with the NMR signals detected by said detecting means In the first echo time in the imaging cycle which is to be the standard, and the NMR signals detected in said first echo time at the imaging cycle subsequent to that of said standard.

Also, in this MRI apparatus, said temperature change distribution image generating means includes means for calculating a standard complex image using the NMR signals detected by said detecting means in said first echo time in the imaging cycle made to be the standard, and as well calculating a complex image using the NMR signals detected by said detecting means in said first echo time in an imaging cycle subsequent to said standard imaging, and means for calculating a complex difference image by calculating the difference between the two complex images calculated by said complex image calculating means.

Further, said temperature change distribution image generating means may include means for correcting for variation of static magnetic field on said complex image.

Further, said morphological image generating means in this MRI apparatus includes means for generating the morphological image of the examined region using the NMR signals detected by said detecting means in said first echo time and those detected in said second echo time in one imaging cycle.

In this MRI apparatus, said image display means includes means for displaying said temperature change distribution image and said morphological image side by side on one display. Further, this image display means may include means for inserting the temperature change distribution image of the examined region into said morphological image displayed on the full screen.

Also, the pulse sequence executed in this MRI apparatus may be a gradient echo type pulse sequence in which an RF pulse is applied one time and then a plurality of readout gradient magnetic fields are applied with alternating polarity. Further, the pulse sequence may be a spin echo type pulse sequence in which a first RF pulse and a second RF pulse which inverts the nuclear spins excited by the first RF pulse are applied, and then a plurality of readout gradient magnetic fields are applied with alternating polarity.

Further, in this MRI apparatus, said control means controls said RF pulse generating means and gradient magnetic field generating means such that the first RF pulse for exciting the nuclear spins and the subsequent second RF pulse for inverting said nuclear spins are applied to generate a spin echo signal in said second echo time, and as well the gradient magnetic fields are applied before or after said spin echo signal is generated and a generate gradient echo signal in said first echo time.

### BRIEF DESCRIPTION OF THE DRAWINGS

FIG. 1 is a block diagram of the structure of an MRI apparatus in an embodiment of the present invention.

FIG. 2 is a timing chart of the pulse sequence in the first example of operation of the MRI apparatus of the present invention.

FIG. 3 is a flow chart showing the process of generating a morphological image and the temperature change distribution image in the first embodiment of the MRI apparatus of the present invention.

FIG. 4(a)-(c) show examples of displaying the morphological image and the temperature change distribution image in the embodiment of the MRI apparatus of the present invention.

FIG. 5 is a timing chart of the pulse sequence in the second example of operation of the MRI apparatus of the present invention.

FIG. 6 is a timing chart of the pulse sequence in the third example of operation of the MRI apparatus of the present invention.

FIG. 7 is a timing chart of the pulse sequence of a conventional gradient echo type for measurement of the temperature distribution.

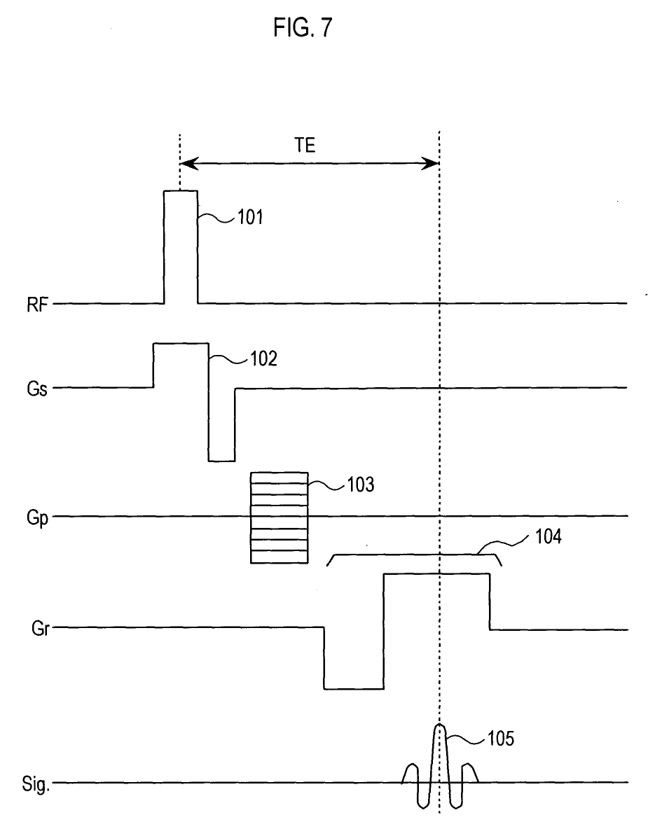

### BEST MODE FOR CARRYING OUT THE INVENTION

Hereinafter, the embodiment of the present invention will be described.

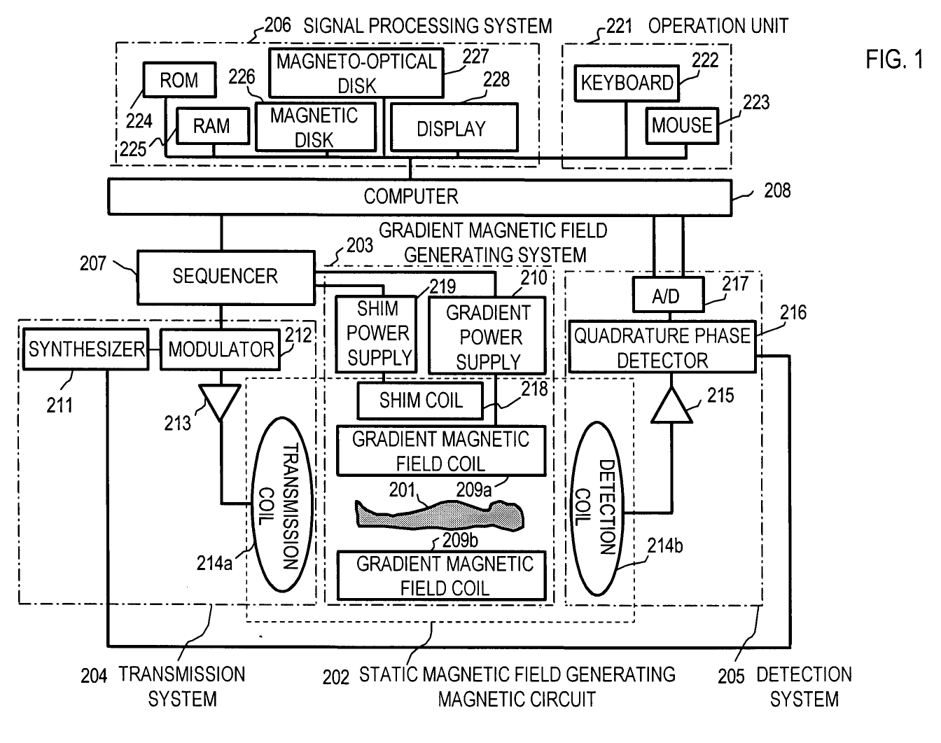

FIG. 1 shows the structure of an MRI apparatus of the present invention. As shown in the figure, the MRI apparatus mainly comprises a static magnetic field generating magnetic circuit 202, a gradient magnetic field generating system 203, a transmission system 204, a detection system 205, a signal processing system 206, a sequencer 207, a computer 208, and an operation unit 221.

The static magnetic field generating magnetic circuit 202 is comprised of a superconductive or resistive electromagnet, or a permanent magnet for generating a uniform static magnetic field Ho in an object 201. In the bore of the magnet a shim coil 218 having a plurality of channels for correcting the non-uniformity of the static magnetic field is placed. Said shimming coil 218 is connected to a shim power supply 219.

The gradient magnetic field generating system 203 is comprised of gradient magnetic field coils 209a and 209b for generating gradient magnetic fields Gx, Gy, and Gz, the intensity of which varies linearly in the x, y, and z directions perpendicular to one another, and a gradient magnetic field power supply 210. This gradient magnetic field generating system 203 provides positional information to the NMR signals generated from the object 201.

The transmission system 204 has a transmitting coil 214a for generating a high frequency magnetic field. In this transmission system 204, the high frequency signal generated by a synthesizer 211 is modulated by a modulator 212, amplified by a power amplifier 213, and provided to the coil 214a in order to apply the high frequency magnetic field to the object 201 and excite nuclear spins (hereinafter referred to as spins) in the object. Although ¹H (Proton) is usually subject to excitation, ³¹P, ¹³C and the like may be also the subject of excitation.

The detection system 205 has a detecting coil 214b for detecting the NMR signals emitted from the object 201. The NMR signals detected by the coil 214b are passed through the amplifier 215, and then Input to the detector 216, in which said signals are made into two series of data by quadrature phase detection. They are then digitalized by the A/D converter 217 and input to the computer 208.

The signal processing system 206 comprises memory devices such as ROM 224, RAM 225, a magnetic disk 226, a magneto-optical disk 227 or the like for memorizing data in the middle of calculation or the final data, that is, the result of the calculation, and a CRT display 228 for displaying the calculation result of the computer 208.

The operation unit 221 is comprised of units for operating input to the computer 208, such as a keyboard 222 and a mouse 223.

The sequencer 207 operates, in accordance with the instruction from the computer 208, the gradient magnetic field generating system 203, the transmission system 204, and the detection system 205 according to the predetermined pulse sequence.

The computer 208 controls said sequencer 308, and as well performs calculation such as two-dimensional Fourier transformation on the two series of data sent from the detection system 205, and generates a morphological image and the temperature change distribution image showing a distribution of the temperature change of the interior of the object, and then, displays them separately or composes them into one image on the display 228.

In this structure, the gradient magnetic field coil 209, the transmitting coil 214a and the detecting coil 214b are placed within the bore of the magnet. The transmitting coil 214a and the detection coil 214b may be one coil for both transmission and reception, or may be the separate coils as shown in the figure.

Hereinafter, the operation of the MRI apparatus thus constructed for generating the morphological image and the temperature change distribution image will be described. For convenience, the direction of the slice-select gradient magnetic field Gs is hereinafter referred to as the z-axis direction, the direction of the phase encoding gradient magnetic field Gp as the y-axis direction, and the direction of the frequency encoding/readout gradient magnetic field Gr as the x-axis direction.

First, the first embodiment will be described.

In this embodiment, for the application of at least one phase encoding gradient magnetic field Gp, the pulse sequence for one slice for generating both a gradient echo signal (or the first echo signal) suitable for obtaining morphological information (anatomic information) and a gradient echo signal (or the second echo signal) suitable for thermometry is repeatedly performed. The morphological image at each time point is generated by the first echo signal, and the temperature change distribution image showing the distribution of temperature change from a standard time set beforehand to a subsequent time is calculated from the second echo signal detected at the standard time and the second echo signal detected at the subsequent time.

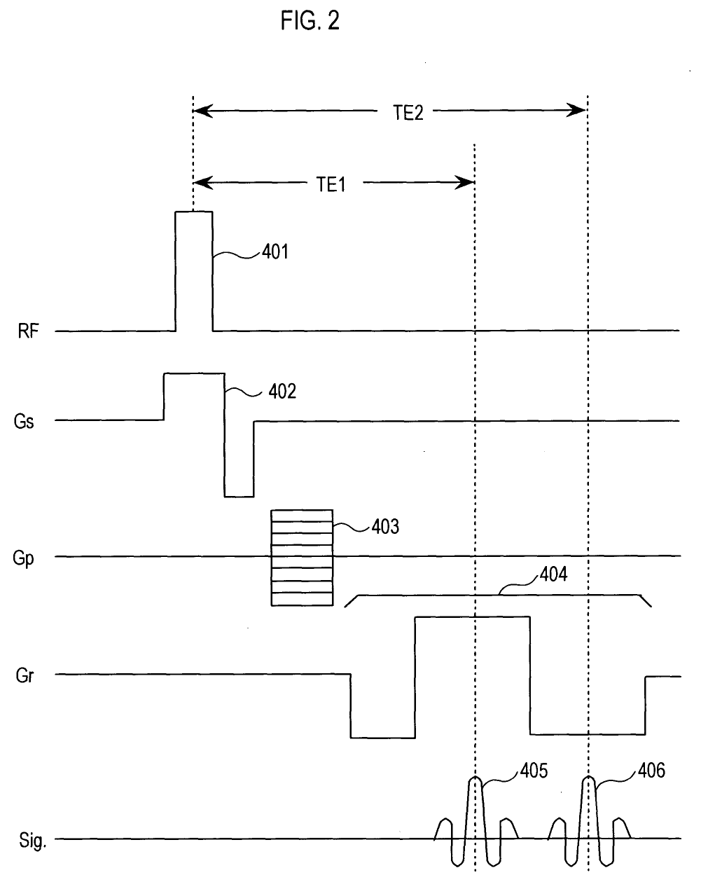

Hereinafter, the details of said operation will be described. First, an example of the multi-echo type pulse sequence for generating at least two gradient echo signals by exciting spins one time and applying only one phase encoding gradient magnetic field Gp will be explained, with reference to FIG. 2. However, this pulse sequence is but an example. The pulse sequence for generating a plurality of gradient echo signals need not be the one shown in the figure, but may instead be any kind of pulse sequence by which a multi echo can be observed when at least one phase encoding gradient magnetic field Gp is applied, such as an SSFP (Steady State Free Precession) type high-speed gradient echo sequence (that is, SSFP sequence) and a GrE type EPI (Echo Planer Imaging) sequence.

In the example of the pulse sequence shown in the figure, the slice-select gradient magnetic field Gs402 selected according to the position in the z direction of the objective slice and a 90° RF pulse RF401 are applied first so as to excite the spins in the slice of thee object. Then, the phase encoding gradient magnetic field Gp403 is applied. Next, the application amount and the polarity of the readout gradient magnetic field Gr404 are controlled such that the gradient echo signal 405 is generated in the echo time TE1 (15 ms, for example) suitable for obtaining the morphological information, thus the phase of the spins is dephased and again rephased. Thus, the echo signal 405 with the echo time TE1 is detected.

Next, the polarity of the readout gradient magnetic field Gr404 is alternated such that the second gradient echo signal 406 is generated in the echo time TE2 (30 ms, for example) suitable for thermometry, and this echo signal 406 in the echo time TE2 is thus detected. Into each of said gradient echo signals obtained by the pulse sequence is encoded the position in the y direction by change of phase by the phase encoding gradient magnetic field Gp 403, and the position in the x direction by change of frequency by the application sequence of the readout gradient magnetic field Gr404.

This pulse sequence is repeated while the intensity of the phase encoding gradient magnetic field Gp403 is varied, for example in 128 levels, so as to obtain the number of gradient echo signals of times TE1 and TE2 respectively required (128) for generating the image of one slice. Hereinafter, the operation for acquiring the required number of the gradient echo signals of times TE1 and TE2 for generating one image for one slice is referred to as one imaging cycle. Such imaging cycle is repeated several times on one slice to generate the morphological image and the temperature distribution image at different times.

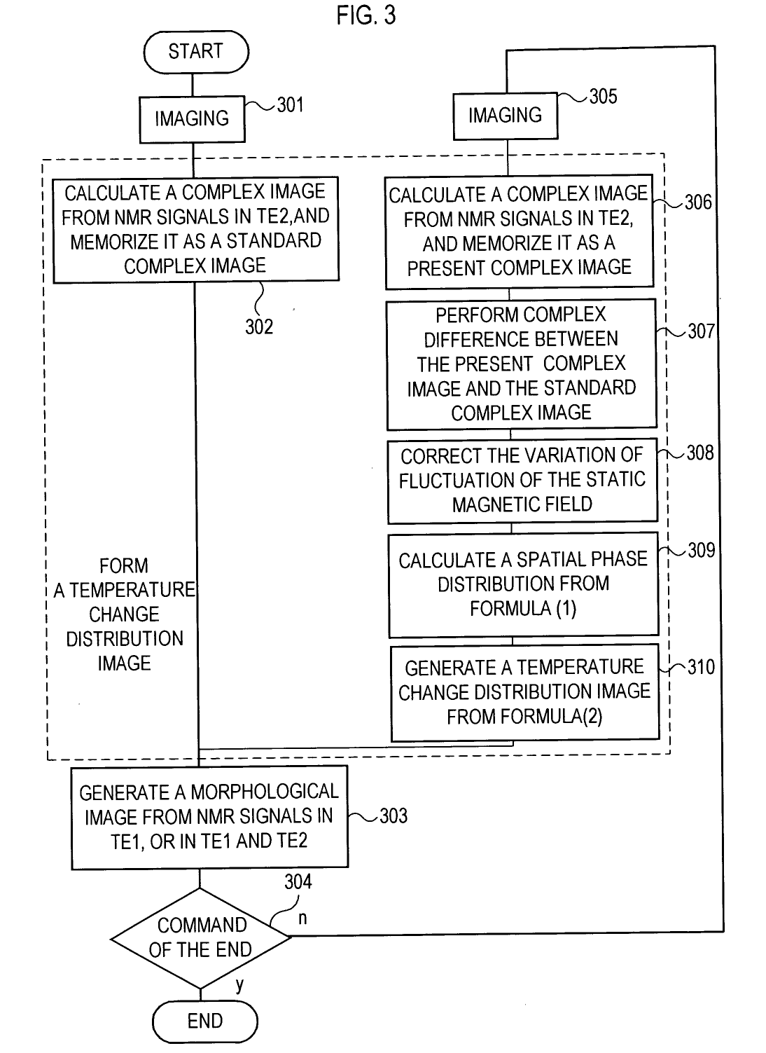

Hereinafter, the details of the operation for generating the morphological image and the temperature distribution image at each time point will be described. FIG. 3 shows the process of forming these images.

First, the computer 208 begins the process shown in FIG. 3 according to the pre-installed program when instructed to begin the thermometry by the operation unit 221, and the first imaging cycle is thus performed. (step 301)

Then, the computer 208 performs two-dimensional Fourier transformation on the echo signal of TE2 obtained in the first imaging cycle to calculate the complex image, and memorizes it as a standard complex image. (step 302)

Next, the computer 208 performs two-dimensional Fourier transformation on the echo signal of TE1 obtained in the first imaging cycle to generate a morphological image (an intensity image) (step 303). Alternatively, the signal obtained by adding the echo signal of TE1 and of TE2 may be used for generating the morphological image, because the S/N ratio can be raised by this addition. However, if the difference between the signals of TE1 and TE2 is large, contrast in a part other than the objective tissue might be large. It is possible to set the apparatus not to perform addition in such a case.

After that, the computer 208 checks whether the end of the measurement is commanded by the operation unit 221 (step 304).

If the end of the thermometry has not been commanded, the process goes on to steps subsequent to the step 305. However, when the thermometry is performed with a predetermined time interval, after it has been verified after it is checked in the step 304 that the end of the thermometry is not instructed, it is better to wait until the next predetermined time for thermometry to go on to steps after the step 305.

In the process of the steps 305 to 309, the computer 208 first performs imaging again in the step 305; performs the two-dimensional Fourier transformation on the echo signal of TE2 for the one slice obtained in this imaging cycle in order to calculate a complex image, which is used as an present complex image (step306). Next, the computer 208 calculates a complex difference image by performing complex difference between the standard complex image previously obtained in the step 302 and the present complex image (step 307).

And, the computer 208 corrects for the variation of fluctuation of the static magnetic field between the previous imaging and this imaging. (step 308)

Next, the computer 208 calculates a spatial phase change distribution by applying to Formula (1) the complex difference image which has been corrected for said variation of fluctuation of the static magnetic field (step 309). Then, the temperature change distribution image is generated by applying to Formula (2) the thus-calculated spatial phase change distribution. (step 310)

This temperature change distribution image indicates the distribution of temperature change within the object between the time point of the first imaging cycle and the time point of the latest imaging cycle.

Next, the computer 208 performs the two-dimensional Fourier transformation on the echo signal of TE1 for one slice obtained in this imaging, or on the signal obtained by adding the echo signal of TE1 and of TE2, to generate a morphological image (an intensity image) (step 303).

The computer 208 repeats the above-described steps until the end of the measurement is instructed, and displays the thus generated morphological image and temperature change distribution image for each time. As a method of displaying these images, it is possible to display the morphological image and the temperature change distribution image side by side, or to superpose the temperature change distribution image on the morphological image.

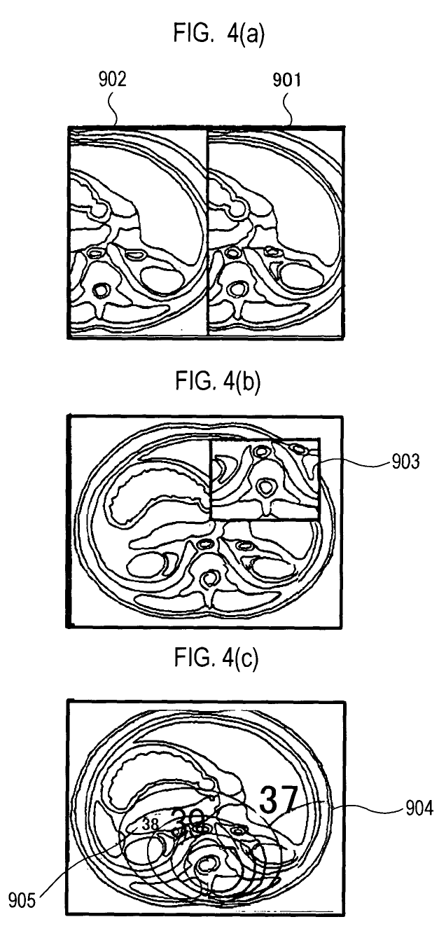

Concretely, as shown in FIG. 4(a), the morphological image 901 can be displayed on the right half of the monitor of the display 228 and the temperature change distribution image 902 is displayed on the left half. It is also possible to put some predetermined colors on the temperature change distribution image to show the temperature change clearly. Also, the morphological image can be displayed on the full screen of the display 228 while the temperature change distribution image 903 is reduced or the image for the region in which the temperature change is calculated is cut out from temperature change distribution image and this cut-out image or reduced image is displayed at a desired position or so as to be movable on the monitor, as shown in FIG. 4(b). Using this method, the morphological image can be largely displayed, and the temperature change distribution image 903 is displayed in a window form at the position which does not disturb observation of the region of interest.

Further, as shown in FIG. 4(c), it is also possible to display the morphological image on the full screen of the display, and as well to overlap on the morphological image the contour lines 904 and numerical values 905 of temperature change distribution calculated from the temperature change distribution image. By employing this method, it is possible to observe both the morphological information (the anatomic information) and the temperature change on one monitor or on one image.

As means for carrying out the above-described embodiment for display, memory for memorizing a plurality of image and means for reading out the plurality of image data memorized in said memory and composing them into one display are required. Since such technique is known in the field of medical apparatus, the explanation of it is omitted.

The morphological image (intensity image) displayed thus qualitatively shows by gradation of light and shade the temperature distribution derived by the signal intensity method. Therefore, it can be understood that the qualitative temperature change based on the signal intensity method and the quantitative temperature change distribution derived by the PPS method are displayed together with the morphological image in the above-described embodiment of display.

In the above-described embodiment, the temperature change distribution is calculated from the spatial phase distribution, which in turn calculated by the complex subtraction of the standard complex image from the present complex image. However, if the equivalent result of it can be obtained, it is also possible, for example to calculate the spatial phase distribution and the temperature distribution of the standard complex image and the present complex image respectively, and use the calculated difference between these two temperature distributions as the temperature change distribution. And, in the process of forming said temperature change distribution, it is also possible to mask the regions other than that of the object. The region of the object can be extracted as a region (x, y) where the absolute value of S(x, y) is equal or above an appropriate threshold, for example 20% above the maximum absolute value of S(x, y). And, in the process of forming the temperature change distribution image, it may be also possible to add an adequate correction such as correction of arc tangent aliasing that is generated by arc tangent operation of Formula (1), besides the correction of the static magnetic field in the step 308.

The above is the description of the first embodiment of the operation for generating the morphological image and the temperature change distribution image performed by the MRI apparatus according to the present invention. Next, the second embodiment of this operation will be described.

In the second embodiment, a multi-echo type pulse sequence in which both the spin echo signal suitable for obtaining morphological information (anatomic information) and the gradient echo signal suitable for thermometry are generated with one excitation of the spins and the application of only one phase encoding gradient magnetic field Gp is used. By this pulse sequence, the spin echo signal and the gradient echo signal for one slice can be obtained at the same time. Similar to the pulse sequence in the first embodiment, such imaging for one slice is time-sequentially repeated. The morphological image is generated from the spin echo signals obtained each time. Further, the temperature change distribution image showing the distribution of temperature change at each time from the standard time point is generated from the gradient echo signals for one slice obtained at the standard time point and those obtained at each time point for one slice.

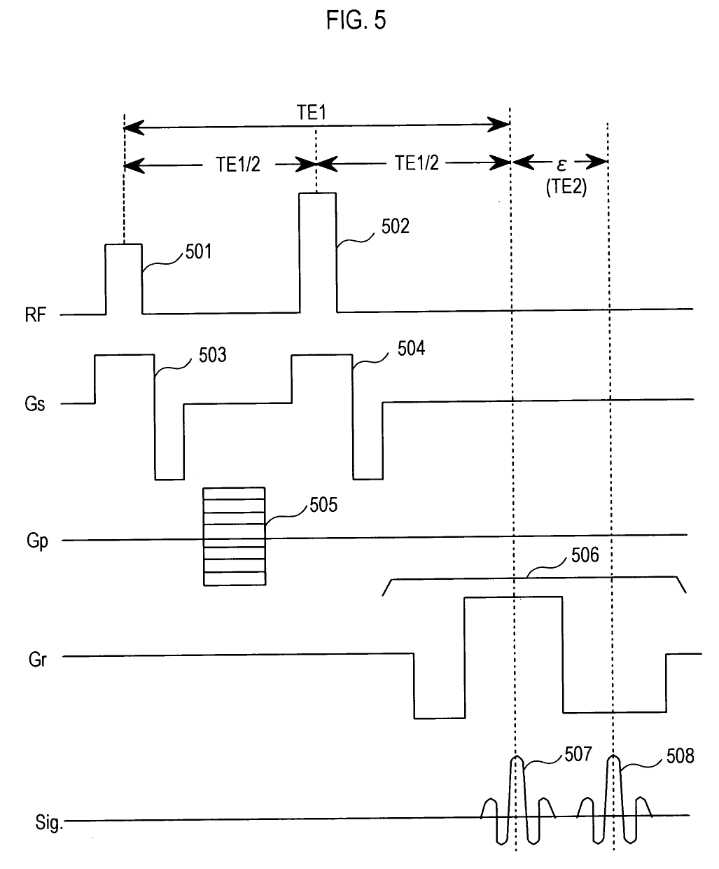

FIG. 5 shows the example of this pulse sequence.

In this pulse sequence, the slice-select gradient magnetic field Gs503 and the 90° RF pulse RF501 selected according to the position of the slice to be taken are applied to excite the nuclear spins in that slice of the object. Then, the phase encoding gradient magnetic field Gp505 is applied. Next, the slice-select gradient magnetic field Gs504 and 180° RF pulse RF502 are applied to invert the nuclear spins in the slice.

Next, the application and the inversion of the readout gradient magnetic field Gr506 is performed such that the spin echo signal 507 is generated when a period of time equal to the time (TE1/2) between the application of the 90° RF pulse RF501 and of the 180° RF pulse RF502 has passed after the application of the 180° RF pulse RF502, that is, when the echo time (TE) has passed after the application of the 90° RF pulse RF 501. Then, the spin echo signals 507 are measured.

Further, the application and the inversion of the readout gradient magnetic field Gr506 are executed after that. When the time ε has passed after the time (TE) when the spin echo 507 is generated, the gradient echo signals 508 are generated and detected.

The above-described pulse sequence is repeatedly executed while the intensity of the phase encoding gradient magnetic field Gp505 is varied enough time to generate the image, for example in 128 levels, and the imaging cycle for one slice is thus performed. The imaging cycle is repeated on the same slice to generate the morphological images and the temperature change distribution images at each time.

In the second embodiment, the morphological image and the temperature change distribution image are generated generally in the same way as in the first embodiment. However, in the step 303 shown in FIG. 3, the morphological image is generated by Fourier-transforming the spin echo signals for one slice. In this case, also, gradient echo signals may be added within to the extent that the quality of the image is not deteriorated.

When the temperature change distribution image is generated in the step 310, the time interval ε between the detection of the spin echo signals and detection of the gradient echo signals used as the TE in Formula (2).

The subsequent steps including the display of the morphological image and the temperature change distribution image are similar to those in the first embodiment.

Next, the third embodiment of the operation for generating the morphological image and the temperature change distribution image performed by the MRI apparatus of the present invention will be described.

As in the second embodiment, the multi-echo pulse sequence in which both the spin echo signals suitable for the acquisition of the morphological information and the gradient echo signals suitable for thermometry are generated during one excitation of the spin and application of only one phase encoding gradient magnetic fields used in the third embodiment. However, in the pulse sequence executed in this embodiment, the spin echo signal suitable for obtaining the morphological information is generated and acquired later than the generation and acquisition of the gradient echo signal suitable for the thermometry. This pulse sequence is suited to obtaining a morphological image emphasizing variation in T2 since it is possible to make TE1 long in this sequence.

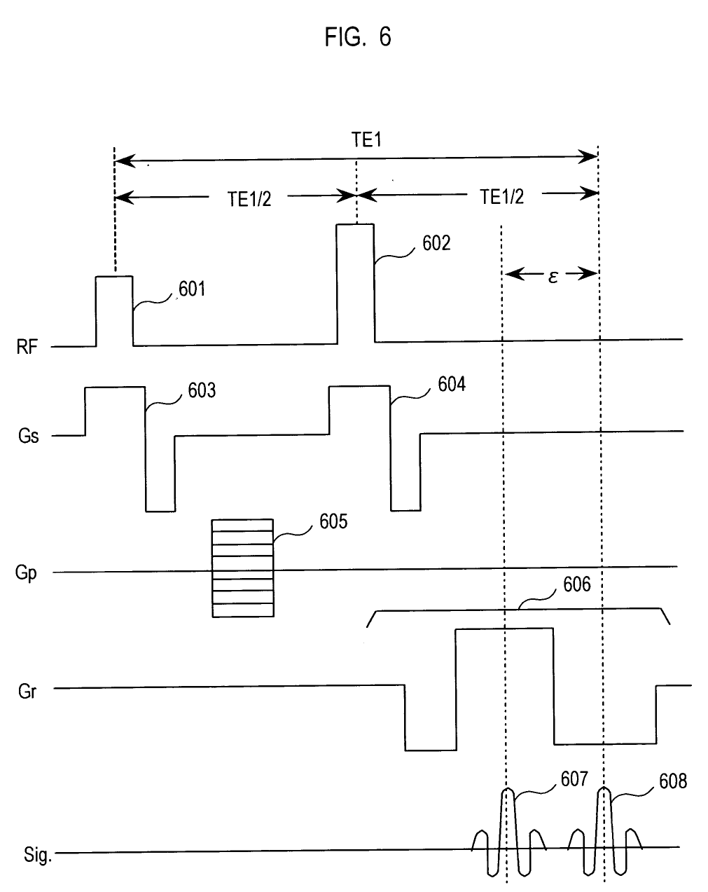

FIG. 6 shows the pulse sequence in the third embodiment. In this pulse sequence, the nuclear spins in the slice of the object are excited at first by applying the slice-select gradient magnetic field Gs603 and the 90° RF pulse RF601 selected in accordance with the position of the objective slice in z direction. Then, the phase encoding gradient magnetic field Gp 605 is applied. Next, the slice-select gradient magnetic field Gs604 and the 180° RF pulse RF602 are applied to invert the nuclear spins in the objective slice.

The spin echo is generated at the point when the half of the echo time TE1 (that is, TE1/2) has been passed since the application of the 180° pulse RF602. Previous to the generation of this spin echo, the application and inversion of the readout gradient magnetic field Gr606 is controlled such that the gradient echo signals 607 are generated and detected ε before the generation of the spin echo.

This pulse sequence is repeatedly executed while the intensity of the phase encoding gradient magnetic field Gp605 is varied enough to generate the image, for example in 128 levels, and the gradient echo signals and the spin echo signals for one slice needed to perform the imaging are thus acquired. Such imaging cycle is repeated on the same slice to generate the morphological image and the temperature change distribution image at each imaging cycle time during the examination.

As in the second embodiment, the morphological image is generated by Fourier-transforming the spin echo signal of TE1 for one slice or the signal made by adding the spin echo signal and the gradient echo signal in the third embodiment. And, when the temperature change distribution image is generated in the step 310, the time interval ε between the detection of the gradient echo signal and detection of the spin echo signal is used as TE in Formula (2). Incidentally, the subsequent steps including display of the morphological image and the temperature change distribution image are similar to those in the first embodiment.

The above is the embodiments of the present invention.

Incidentally, the above-described embodiments are the cases where the temperature change distribution of a period of time is calculated and used as the temperature change distribution image. However, the temperature distributions at each time may be used instead of said temperature change distribution.

As mentioned above, in the pulse sequence employed in the embodiment according to the present invention, both the echo signal, the echo time of which is suitable for obtaining morphological information and the echo signals, the echo time of which is suitable for thermometry are acquired. Thus, both a precise temperature change or temperature change distribution by the PPS method and the fine morphological image, the S/N ratio of which is high can be obtained. That is, since the echo signals suitable for obtaining the morphological information and the echo signal suitable for thermometry are generated in a common pulse sequence, the morphological image and the temperature distribution or the temperature change distribution can be preferably obtained more rapidly and with less process load, in comparison with the case where both signals are acquired separately in the independent pulse sequences.

Therefore, both the morphological image and the temperature distribution or the temperature change distribution can be obtained preferably and efficiently.

What is claimed is:

1. A magnetic resonance imaging apparatus comprising:
static magnetic field generating means for generating a static magnetic field in a space in which an object to be examined is laid;
RF pulse generating means for applying an RF pulse to generate nuclear magnetic resonance in nuclear spins existing in an examined region of said object laid in the static magnetic field;
gradient magnetic field generating means for applying on said object a plurality of gradient magnetic fields including a phase encoding gradient magnetic field to phase-encode NMR signals generated from said examined region;
control means for controlling the application of said RF pulse and gradient magnetic fields to repeatedly execute a pulse sequence for generating a plurality of NMR signals having different echo times and encoded with the same phase after exciting said nuclear spins one time;
detecting means for detecting the plurality of NMR signals generated from said examined region with different respective echo times;
temperature distribution image generating means for generating a temperature distribution image of said examined region by using the NMR signals detected by said detecting means in a first echo time;
morphological image generating means for generating a morphological image of said examined region by using the NMR signals detected by said detecting means in a second echo time; and
image display means for displaying said temperature distribution image and said morphological image.

2. A magnetic resonance imaging apparatus according to claim 1, wherein said temperature distribution image generating means includes means for making an image of the temperature distribution of said examined region in accordance with a spatial phase distribution that is calculated with the NMR signals detected by said detecting means in said first echo time.

3. A magnetic resonance imaging apparatus according to claim 1, wherein said morphological image generating means includes means for generating the morphological image of said examined region by using the NMR signals detected by said detecting means in said first echo time and in said second echo time.

4. A magnetic resonance imaging apparatus according to claim 1, wherein said image display means includes means for displaying said temperature distribution image and morphological image side by side on one display screen.

5. A magnetic resonance imaging apparatus according to claim 2, wherein said image display means includes means for inserting the temperature distribution in said examined region or an image depicting temperature distribution in a region in which the temperature distribution is measured into said morphological image displayed on the full screen, and displaying the inserted region.

6. A magnetic resonance imaging apparatus according to claim 1, wherein said pulse sequence is a gradient echo type pulse sequence, in which an RF pulse is applied one time and a plurality of readout gradient magnetic fields are successively applied with alternating polarity.

7. A magnetic resonance imaging apparatus according to claim 1, wherein said pulse sequence is a spin echo type pulse sequence in which a first RF pulse and a second RF pulse for inverting the nuclear spins excited by said first RF pulse, and a plurality of readout gradient magnetic fields are successively applied with alternating polarity.

8. A magnetic resonance imaging apparatus comprising:
static magnetic field generating means for generating a static magnetic field in a space in which an object is laid;
RF pulse generating means for applying an RF pulse to generate nuclear magnetic resonance in nuclear spins in the region of said object to be examined;
gradient magnetic fields generating means for applying a plurality of gradient magnetic fields including a phase encoding gradient magnetic fields to phase-encode the NMR signals generated from said region;
control means for controlling the application of said RF pulse and said gradient magnetic fields to repeatedly execute the pulse sequence for generating a plurality of NMR signals having different echo times and encoded with the same phase after exciting said nuclear spins one time, in order to time-sequentially perform imaging plural times on said region of the object;
detecting means for detecting the plurality of NMR signals having different echo times generated from said examined region;
temperature change distribution image generating means for calculating a temperature distribution in said examined region at each time point by using the NMR signals detected by said detecting means in a first echo time, and generating a temperature change distribution image of said examined region by comparing one temperature distribution and another one;
morphological image generating means for generating a morphological image of said examined region by using the NMR signals detected by said detecting means in a second echo time in said one imaging;
image display means for displaying said temperature change distribution image and said morphological image.

9. A magnetic resonance imaging apparatus according to claim 8, wherein said temperature change distribution image generating means includes means for making an image of the temperature change distribution in said examined region in accordance with a spatial phase distribution that is calculated with the NMR signals detected by said detecting means in said first echo time in the imaging cycle chosen to be the standard, and in an imaging cycle subsequent to this standard imaging.

10. A magnetic resonance imaging apparatus according to claim 9, wherein said temperature change distribution image generating means includes means for calculating a standard complex image with the NMR signals detected by said detecting means in said first echo time in the standard imaging cycle, and as well calculating a complex image with the NMR signals detected by said detecting means in said first echo time in the imaging subsequent to said standard imaging, and means for calculating a complex difference image by calculating the difference between the two complex images calculated by said complex image calculating means.

11. A magnetic resonance imaging apparatus according to claim 10, wherein said temperature change distribution image generating means further includes means for correcting for fluctuation of the static magnetic field in said complex difference image cycle.

12. A magnetic resonance imaging apparatus according to claim 8, wherein said morphological image generating means includes means for generating the morphological image of said examined region by using the NMR signals detected by said detecting means in said first and said second echo time in one imaging.

13. A magnetic resonance imaging apparatus according to claim 8, wherein said image display means includes means for displaying said temperature change distribution image and said morphological image side by side on one display screen.

14. A magnetic resonance imaging apparatus according to claim 13, wherein said image display means includes means for inserting the temperature distribution or an image depicting temperature distribution in a region in which the temperature distribution is measured in said morphological image displayed on the full screen and displaying the inserted image.

15. A magnetic resonance imaging apparatus according to claim 8, wherein said pulse sequence is a gradient echo type pulse sequence in which an RF pulse is applied one time and a plurality of readout gradient magnetic fields are applied successively with alternating polarity.

16. A magnetic resonance imaging apparatus according to claim 8, wherein said pulse sequence is a spin echo type pulse sequence in which a first RF pulse, a second RF pulse to invert the nuclear spins excited by said first RF pulse, and the plurality of readout gradient magnetic fields are applied successively with alternating polarity.

17. A magnetic resonance imaging apparatus according to claim 16, wherein said control means applies said first RF pulse to excite the nuclear spins, and said second RF pulse to invert said spins so as to generate the spin echo signal in said second echo time, and as well, controls said RF pulse generating means and gradient magnetic field generating means to apply the gradient magnetic fields before or after the generation of said spin echo signals so as to generate the gradient echo signal in said first echo time.
